(19) United States
(12) Patent Application Publication
(10) Pub. No.: US 2008/0070825 A1
(43) Pub. Date: Mar. 20, 2008

(54) 2-Methyl-2-alkenyl-substituted 1,3-dioxanes as odoriferous substances

(75) Inventors: Heinz-Jurgen Bertram, Holzminden (DE); Thomas Obrocki, Pattensen (DE); Sonja Luders, Holzminden (DE)

Correspondence Address: ROYLANCE, ABRAMS, BERDO & GOODMAN, L.L.P., 1300 19TH STREET, N.W., SUITE 600, WASHINGTON,, DC, 20036, US

(73) Assignee: SYMRISE GMBH & CO. KG, Holzminden (DE)

(21) Appl. No.: 11/854,854

(22) Filed: Sep. 13, 2007

(30) Foreign Application Priority Data
Sep. 16, 2006 (DE) 10 2006 043 587.7

(51) Int. Cl. A61K 8/33; A61Q 13/00; C07D 319/06

(52) U.S. Cl. 512/12; 549/369

(57) ABSTRACT

The present invention relates to a compound of the formula (I) 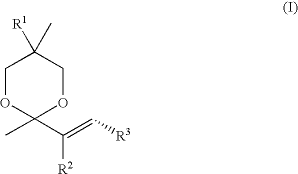 wherein

R¹ denotes hydrogen or methyl, and R² and R³ independently of one another denote methyl or ethyl.

The present invention primarily relates to certain novel compounds of the formula (I) (2-methyl-2-alkenyl-substituted 1,3-dioxanes), a process for their preparation and the use of these compounds as an odoriferous substance. In addition, the invention relates inter alia to perfumed products, such as odoriferous substance mixtures (odoriferous substance compositions) comprising a (sensorially active) amount of these compounds. Further aspects of the invention emerge from the following description and the attached patent claims.

The novel compounds (2-methyl-2-alkenyl-substituted 1,3-dioxanes) of the formula (I) have the following structure:

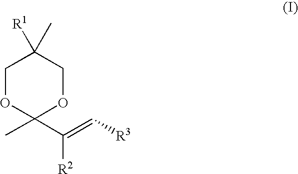

wherein

R¹ denotes hydrogen or methyl, and

R² and R³ independently of one another denote methyl or ethyl.

The wavy line in the above and the further following structural formulae means that the double bond can be in the (E) or (Z) configuration.

In the perfume industry there is generally a need for novel and original odoriferous substances, since novel and modern fragrances are constantly to be made available to consumers. Because of the consumer's increasing demand for novel modern fragrance notes, in the perfume industry there is a constant need for novel fragrances with which novel effects can be achieved in perfumes and new fashion trends can be created in this manner.

For creation of novel modern compositions, there is a constant need for novel odoriferous substances which have particular olfactory properties and are suitable for serving as a basis for composition of novel modern perfumes. The odoriferous substances sought should have further notes and aspects, in addition to a typical primary smell, which impart to them olfactory character, such as, for example, freshness and complexity.

It was therefore the object of the present invention to discover odoriferous substances which have novel fresh olfactory properties and with which particular olfactory notes and aspects can be imparted to odoriferous substance compositions.

In addition, odoriferous substances which increase the intensity of the olfactory perception of other odoriferous substances (that is to say function as boosters) are of great interest for perfumistic composition.

The search for suitable odoriferous substances which led to the present invention was made difficult by the following circumstances:
The mechanisms of olfactory perception are not adequately known.
The relationships between the specific olfactory perception on the one hand and the chemical structure of the associated odoriferous substance on the other hand have not been adequately researched.
Slight changes in the structural make-up of a known odoriferous substance often already have the effect of marked changes in the sensorial properties and impair the tolerability for the human organism.

Success in the search for suitable odoriferous substances therefore depends greatly on the intuition of the searcher.

It has now been found, surprisingly, that 2-methyl-2-alkenyl-substituted 1,3-dioxanes of the above formula (I) are suitable for achieving the stated object.

In the opinion of perfumers, the 2-methyl-2-alkenyl-substituted 1,3-dioxanes of the formula (I) according to the invention have the following complex olfactory characteristics:

minty, camphorous, damascone-like, fruity, rum and raisin, dried fruit, sweet, green.

“Rum and raisin” is understood generally as the alcohol smell of raisins steeped in rum.

“Minty” can vary somewhat from peppermint to spearmint, in some cases paired with a light eucalyptus note, depending on the particular meaning of the radicals R¹, R² and R³.

The sought-after damascone-like note of the compounds according to the invention is most similar to that of alpha-damascone.

It has been found, surprisingly, that the 2-methyl-2-alkenyl-substituted 1,3-dioxanes of the formula (I) differ significantly in olfactory properties from the structurally related compounds (substituted 1,3-dioxanes) described in the literature.

EP 0 039 029 describes 2-(1′-methylbutyl)-1,3-dioxanes substituted by 2 to 6 alkyl groups in positions four and five. These compounds are attributed a flowery and fruity note.

U.S. Pat. No. 4,146,506 relates to perfume compositions with 4-isopropyl-5,5-dimethyl-1,3-dioxane having C1-C3 alkyl substituents in position two. These 1,3-dioxanes are described with a fruity, herbal and woody note.

U.S. Pat. No. 5,888,961 describes 2-isobutyl-5-methyl-1,3-dioxane with an intense olfactory impression of camomile and fruity, aniseed-like, minty, camphorous and green notes.

CH 592649 relates to 2-(2′-methyl-1′-propenyl)-1,3-dioxanes with one or more methyl or ethyl groups in position four and/or five and/or six. These are attributed a rose-like olfactory impression similar to rose oxide. In CH 592649, 5,5-dimethyl-2-(2′-methyl-1′-propenyl)-1,3-dioxane is structurally the most similar to the compounds of the formula (I) according to the invention and, according to CH 592649, has a thyme smell.

Pishch. Prom-st. (Moscow) (1990), (2), 54-57 describes 2-isobutyl-2,5,5-trimethyl-1,3-dioxane as a compound having a woody-fruity smell with a solvent note.

The odoriferous substances having a substituted 1,3-dioxane structure described to date in the literature are odoriferous substances which, compared with the compounds of the formula (I) according to the invention,
have different olfactory properties, and/or
differ significantly in respect of their substitution pattern in positions 2, 4, 5 and/or 6, and/or
differ in respect of the chain length and/or structure of the side chain at position 2.

Furthermore, 1,3-dioxanes which are only monosubstituted at position 2 (i.e. are compounds which are not according to the invention and are derived from aldehydes) are less stable than compounds disubstituted at position 2 (such as e.g. the compounds according to the invention and other compounds, which are derived from ketones).

In our own investigations it has furthermore been found that compared with compounds according to the invention, the olfactory impression is changed markedly in the absence of a substituent at position 5.

Thus, for example, in contrast to the compounds of the formula (I) according to the invention, the compound 2-methyl-2-(1-methyl-propenyl)-1,3-dioxane, which is not according to the invention, smells fishy, walnut-like, herbal, flowery, with notes of laurel, lavender and linalool.

Preferred compounds of the formula (I) are those where R²=methyl, i.e. compounds of the formula (II):

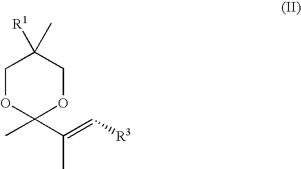

wherein

R¹ denotes hydrogen or methyl, and

R³ denotes methyl or ethyl.

Compounds of the formula (I) according to the invention and in particular compounds of the formula (II) according to the invention in which the double bond has the (E) configuration are preferred.

Particularly preferred compounds of the formula (I) according to the invention are those where R²=methyl and R³=methyl, the double bond preferably having the (E) configuration. These particularly preferred compounds according to the invention are those of the formula (III).

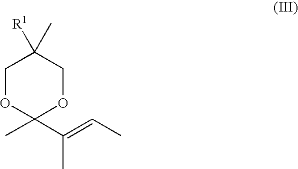

wherein

R¹ denotes hydrogen or methyl.

The (E) isomers of the formula (III) according to the invention display a particularly pronounced and very natural, bright and transparent smell.

In the case where R¹ denotes hydrogen, the compounds of the formula (I) according to the invention and in particular the compounds of the formulae (II) and (III) according to the invention, particularly preferably the compounds according to the invention having the (E) configuration at the double bond, can be in the form of the cis or trans diastereomer or also in the form of any desired mixture of these diastereomers.

In mixtures with other odoriferous substances, the compounds of the formula (I) according to the invention are already capable in a low dosage of increasing the intensity of an odoriferous substance mixture and of rounding off the olfactory picture of the odoriferous substance mixture, and of imparting to the mixture more radiance and naturalness. In higher dosages, the fresh and damascone-like smell takes effect in particular.

The present invention also relates to mixtures comprising or consisting of:
(a) one or more compounds of the formula (I) according to the invention, but preferably of the formula (II), particularly preferably of the formula (III), very particularly preferably having the (E) configuration at the double bond,

and
(b) 2-ethyl-2,5,5-trimethyl-1,3-dioxane of the formula (IV)

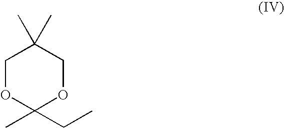

and/or
(c) 3-methyl-3-penten-2-one of the formula (V)

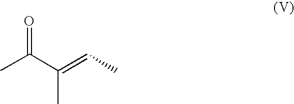

and optionally one, two or more further odoriferous substances.

2-Ethyl-2,5,5-trimethyl-1,3-dioxane of the formula (IV) is known from Pishch. Prom-st. (Moscow) 1990, (2), 54-57.

3-Methyl-3-penten-2-one of the formula (V) is known from Perfumer & Flavorist 2001, 26(2), 16-21.

Mixtures according to the invention which comprise constituents (a) and (b) and/or (c) have a more intensified damascone note compared with a compound of the formula (I); moreover, the olfactory impression of such a mixture is overall even fresher, fruitier, more radiant and more complex than the olfactory impression of a compound of the formula (I) per se. The same applies accordingly to the comparison between mixtures which on the one hand comprise only one compound of the formula (I) in addition to one, two or more further odoriferous substances, and on the other hand comprise, in addition to one, two or more further odoriferous substances, not only a compound of the formula (I) as constituent (a) but additionally also a compound of the formula (IV) and/or a compound of the formula (V) as constituent (b) and/or (c).

A preferred mixture according to the invention comprises or consists of:
(a) 78.0 to 99.9 parts by weight in total of one or more compounds according to one of claims 1 to 5,

and
(b) 0.25 to 20 parts by weight of 2-ethyl-2,5,5-trimethyl-1,3-dioxane of the formula (IV)

and/or
(c) 0.1 to 3.0 parts by weight of 3-methyl-3-penten-2-one of the formula (V),

wherein the sum of constituents (a), (b) and (c) is 100 parts by weight.

In addition, one, two or more further odoriferous substances are optionally also present.

A particularly preferred mixture according to the invention comprises or consists of:

(a) 91 to 99.75 parts by weight of one or more compounds according to one of claims 1 to 5,

and

(b) 0.5 to 8 parts by weight of 2-ethyl-2,5,5-trimethyl-1,3-dioxane of the formula (IV)

and/or

(c) 0.25 to 1 part by weight of 3-methyl-3-penten-2-one of the formula (V), wherein the sum of constituents (a), (b) and (c) is 100 parts by weight, and optionally one, two or more further odoriferous substances.

The present invention also relates to the use of a compound of the formula (I) according to the invention, that is to say preferably a compound of the formula (II) and particularly preferably a compound of the formula (III), the double bond in each case particularly preferably having the (E) configuration, as an odoriferous substance or booster.

The present invention accordingly also relates to the use of a mixture according to the invention which comprises the constituents (a) and (b) and/or (c) specified above and optionally one, two or more further odoriferous substances as an odoriferous substance mixture or perfumed product.

The preparation of the 2-methyl-2-alkenyl-substituted 1,3-dioxanes of the formula (I) according to the invention is carried out, for example, by generally known methods of organic synthesis by means of reaction (ketalization) of the corresponding ketone of the formula (K) (having radicals R² and R³) with a corresponding aliphatic 1,3-diol of the formula (D) (having the radical R¹), as described in the equation below:

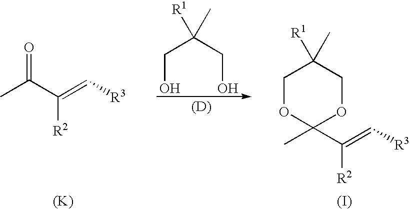

wherein

R¹, R² and R³ in each case have the abovementioned meaning, preferably a meaning described above as preferred.

The configuration of the double bond of the ketone (K) is retained when the ketalization is carried out; the use of ketones having (E) configurations at the double bond is thus preferred.

The ketalization is preferably carried out in the presence of an acid catalyst, such as, for example, p-toluenesulfonic acid (cf. T, Eicher, L. F. Tietze, Organisch-chemisches Grundpraktikum [Basic Practical Organic Chemistry], Georg Thieme Verlag Stuttgart, 1993, 198), but other acids employed for ketalization reactions, such as methanesulfonic acid or sulfuric acid, are of course also alternatively suitable.

Preferably, at least 1.2 molar equivalents of diol (D), based on the ketone (K) employed, are employed in the ketalization reaction. The reaction is preferably carried out in an inert hydrocarbon, such as, for example, toluene or cyclohexane, as a diluent. The reaction temperature is preferably in the range of from 70 to 140° C. In this context, the reaction time is regularly 4 to 20 hours.

The ketones (K) can be obtained commercially, as in the case of the (preferably (E)-configured) 3-methyl-3-penten-2-one of the formula (V), or prepared by condensation of 2-butanone with the corresponding aldehyde in the presence of potassium hydroxide as a catalyst (e.g. in accordance with J. Chem. Soc. (1944), 66, 1517-1519).

The 1,3-diols (D) employed in the ketalization reaction are commercially obtainable.

The present invention also relates to a perfumed product comprising

(A) a solid or semi-solid carrier

and

a sensorially active amount, in contact with the solid or semi-solid carrier, of a
compound of the formula (I) according to the invention, preferably of the formula (II), particularly preferably of the formula (III), particularly advantageously having the (E) configuration at the double bond,

or
mixture according to the invention comprising the abovementioned constituents (a) and (b) and/or (c) and optionally one, two or more further odoriferous substances,

or

(B) a liquid phase

and

dissolved or suspended therein or diluted therewith, a sensorially active amount of a
compound of the formula (I) according to the invention, preferably of the formula (II), particularly preferably of the formula (III), particularly advantageously having the (E) configuration at the double bond

or
mixture according to the invention comprising constituents (a) and (b) and/or (c) and optionally one, two or more further odoriferous substances.

Conventional other perfume constituents with which the compounds of the formula (I) according to the invention and the mixtures according to the invention which additionally comprise the abovementioned constituents (b) and/or (c) can advantageously be combined to form an odoriferous substance mixture (=perfume oil composition) are to be found e.g. in Steffen Arctander, Perfume and Flavor Chemicals, private publishing house, Montclair, N. J., 1969; K. Bauer, D. Garbe, H. Surburg, Common Fragrance and Flavor Materials, 4th Edition, Wiley-VCH, Weinheim 2001.

There may be mentioned in detail:

extracts from natural raw materials, such as essential oils, concretes, absolutes, resins, resinoids, balsams, tinctures, such as e.g. amber tincture; amyris oil; angelica seed oil; angelica root oil; aniseed oil; valerian oil; basil oil; tree moss absolute; bay oil; artemisia oil; benzoin resin; bergamot oil; beeswax absolute; birch tar oil; bitter almond oil; bean leaf oil; buchu leaf oil; cabreuva oil; cade oil; calamus oil; camphor oil; cananga oil; cardamom oil; cascarilla oil; cassia oil; cassia absolute; castoreum absolute; cedar leaf oil; cedar wood oil; cistus oil; citronella oil; lemon oil; copaiva balsam; copaiva balsam oil; coriander oil; costus root oil; cumin oil; cypress oil; davana oil; dill weed oil; dill seed oil; eau de brouts absolute; oak moss absolute; elemi oil; tarragon oil; Eucalyptus citriodora oil; eucalyptus oil; fennel oil; spruce needle oil; galbanum oil; galbanum resin; geranium oil; grapefruit oil; guaiac wood oil; gurjunene balsam; gurjunene balsam oil; helichrysum absolute; helichrysum oil; ginger oil; iris root absolute; iris root oil; jasmine absolute; calamus oil; camomile oil blue; camomile oil Roman; carrot seed oil; cascarilla oil; pine needle oil; spearmint oil; caraway oil; labdanum oil; labdanum absolute; labdanum resin; lavandin absolute; lavandin oil; lavender absolute; lavender oil; lemon grass oil; lovage oil; lime oil distilled; lime oil pressed; linaloa oil; Litsea cubeba oil; bay leaf oil; mace oil; marjoram oil; mandarin oil; massoi bark oil; mimosa absolute; musk seed oil; musk tincture; muscatel sage oil; nutmeg oil; myrrh absolute; myrrh oil; myrtle oil; clove leaf oil; clove blossom oil; neroli oil; olibanum absolute; olibanum oil; opopanax oil; orange blossom absolute; orange oil; oregano oil; palmarosa oil; patchouli oil; perilla oil; Peru balsam oil; parsley leaf oil; parsley seed oil; petitgrain oil; peppermint oil; pepper oil; pimento oil; pine oil; poley oil; rose absolute; rose wood oil; rose oil; rosemary oil; sage oil Dalmatian; sage oil Spanish; sandalwood oil; celery seed oil; spike lavender oil; star aniseed oil; styrax oil; tagetes oil; fir needle oil; tea tree oil; turpentine oil; thyme oil; tolu balsam; tonka absolute; tuberose absolute; vanilla extract; violet leaf absolute; verbena oil; vetiver oil; juniper berry oil; wine yeast oil; wormwood oil; wintergreen oil; ylang oil; hyssop oil; civet absolute; cinnamon leaf oil; cinnamon bark oil; and fractions thereof or constituents isolated therefrom;

individual odoriferous substances from the group consisting of the hydrocarbons, such as e.g. 3-carene; α-pinene; β-pinene; α-terpinene; γ-terpinene; p-cymene; bisabolene; camphene; caryophyllene; cedrene; farnesene; limonene; longifolene; myrcene; ocimene; valencene; (E,Z)-1,3,5-undecatriene;

the aliphatic alcohols, such as e.g. hexanol; octanol; 3-octanol; 2,6-dimethylheptanol; 2-methyl-2-heptanol; 2-methyl-2-octanol; (E)-2-hexenol; (E)- and (Z)-3-hexenol; 1-octen-3-ol; mixture of 3,4,5,6,6-pentamethyl-3/4-hepten-2-ol and 3,5,6,6-tetramethyl-4-methyleneheptan-2-ol; (E,Z)-2,6-nonadienol; 3,7-dimethyl-7-methoxyoctan-2-ol; 9-decenol; 10-undecenol; 4-methyl-3-decen-5-ol;

the aliphatic aldehydes and acetals thereof, such as e.g. hexanal; heptanal; octanal; nonanal; decanal; undecanal; dodecanal; tridecanal; 2-methyloctanal; 2-methylnonanal; (E)-2-hexenal; (Z)-4-heptenal; 2,6-dimethyl-5-heptenal; 10-undecenal; (E)-4-decenal; 2-dodecenal; 2,6,10-trimethyl-5,9-undecadienal; heptanal diethyl acetal; 1,1-dimethoxy-2,2,5-trimethyl-4-hexene; citronellyloxyacetaldehyde;

the aliphatic ketones and oximes thereof, such as e.g. 2-heptanone; 2-octanone; 3-octanone; 2-nonanone; 5-methyl-3-heptanone; 5-methyl-3-heptanone oxime; 2,4,4,7-tetramethyl-6-octen-3-one; the aliphatic sulfur-containing compounds, such as e.g. 3-methylthiohexanol; 3-methylthiohexyl acetate; 3-mercaptohexanol; 3-mercaptohexyl acetate; 3-mercaptohexyl butyrate; 3-acetylthiohexyl acetate; 1-menthene-8-thiol;

the aliphatic nitriles, such as e.g. 2-nonenoic acid nitrile; 2-tridecenoic acid nitrile; 2,12-tridecenoic acid nitrile; 3,7-dimethyl-2,6-octadienoic acid nitrile; 3,7-dimethyl-6-octenoic acid nitrile;

the aliphatic carboxylic acids and esters thereof, such as e.g. (E)- and (Z)-3-hexenyl formate; ethyl acetoacetate; isoamyl acetate; hexyl acetate; 3,5,5-trimethylhexyl acetate; 3-methyl-2-butenyl acetate; (E)-2-hexenyl acetate; (E)- and (Z)-3-hexenyl acetate; octyl acetate; 3-octyl acetate; 1-octen-3-yl acetate; ethyl butyrate; butyl butyrate; isoamyl butyrate; hexyl butyrate; (E)- and (Z)-3-hexenyl isobutyrate; hexyl crotonate; ethyl isovalerate; ethyl 2-methylpentanoate; ethyl hexanoate; allyl hexanoate; ethyl heptanoate; allyl heptanoate; ethyl octanoate; ethyl (E,Z)-2,4-decadienoate; methyl 2-octynate; methyl 2-nonynate; allyl 2-isoamyloxyacetate; methyl 3,7-dimethyl-2,6-octadienoate;

the acyclic terpene alcohols, such as e.g. citronellol; geraniol; nerol; linalool; lavadulol; nerolidol; farnesol; tetrahydrolinalool; tetrahydrogeraniol; 2,6-dimethyl-7-octen-2-ol; 2,6-dimethyloctan-2-ol; 2-methyl-6-methylen-7-octen-2-ol; 2,6-dimethyl-5,7-octadien-2-ol; 2,6-dimethyl-3,5-octadien-2-ol; 3,7-dimethyl-4,6-octadien-3-ol; 3,7-dimethyl-1,5,7-octatrien-3-ol; 2,6-dimethyl-2,5,7-octatrien-1-ol; and formates, acetates, propionates, isobutyrates, butyrates, isovalerates, pentanoates, hexanoates, crotonates, tiglinates, 3-methyl-2-butenoates thereof;

the acyclic terpene aldehydes and ketones, such as e.g. geranial; neral; citronellal; 7-hydroxy-3,7-dimethyloctanal; 7-methoxy-3,7-dimethyloctanal; 2,6,10-trimethyl-9-undecenal; geranylacetone; and the dimethyl and diethyl acetals of geranial, neral, 7-hydroxy-3,7-dimethyloctanal;

the cyclic terpene alcohols, such as e.g. menthol; isopulegol; alpha-terpineol; terpinen-4-ol; menthan-8-ol; menthan-1-ol; menthan-7-ol; borneol; isoborneol; linalool oxide; nopol; cedrol; ambrinol; vetiverol; guaiol; and formates, acetates, propionates, isobutyrates, butyrates, isovalerates, pentanoates, hexanoates, crotonates, tiglinates, 3-methyl-2-butenoates thereof;

the cyclic terpene aldehydes and ketones, such as e.g. menthone; isomenthone; 8-mercaptomenthan-3-one; carvone; camphor; fenchone; alpha-ionone; beta-ionone; alpha-n-methylionone; beta-n-methylionone; alpha-isomethylionone; beta-isomethylionone; alpha-irone; alpha-damascone; beta-damascone; beta-damascenone; delta-damascone; gamma-damascone; 1-(2,4,4-trimethyl-2-cyclohexen-1-yl)-2-buten-1-one; 1,3,4,6,7,8a-hexahydro-1,1,5,5-tetramethyl-2H-2,4a-methanonaphthalen-8(5H)-one; nootkatone; dihydronootkatone; alpha-sinensal; beta-sinensal; acetylated cedar wood oil (methyl cedryl ketone);

the cyclic alcohols, such as e.g. 4-tert-butylcyclohexanol; 3,3,5-trimethylcyclo-hexanol; 3-isocamphylcyclohexanol; 2,6,9-trimethyl-Z2,Z5,E9-cyclododecatrien-1-ol; 2-isobutyl-4-methyltetrahydro-2H-pyran-4-ol;

the cycloaliphatic alcohols, such as e.g. alpha,3,3-trimethylcyclohexylmethanol; 2-methyl-4-(2,2,3-trimethyl-3-cyclopent-1-yl)butanol; 2-methyl-4-(2,2,3-trimethyl-3-cyclopent-1-yl)-2-buten-1-ol; 2-ethyl-4-(2,2,3-trimethyl-3-cyclopent-1-yl)-2-buten-1-ol; 3-methyl-5-(2,2,3-trimethyl-3-cyclopent-1-yl)-pentan-2-ol; 3-methyl-5-(2,2,3-trimethyl-3-cyclopent-1-yl)-4-penten-2-ol; 3,3-dimethyl-5-(2,2,3-trimethyl-3-cyclopent-1-yl)-4-penten-2-ol; 1-(2,2,6-trimethylcyclohexyl)pentan-3-ol; 1-(2,2,6-trimethylcyclohexyl)hexan-3-ol;

the cyclic and cycloaliphatic ethers, such as e.g. cineol; cedryl methyl ether; cyclododecyl methyl ether; (ethoxymethoxy)cyclododecane; alpha-cedrene epoxide; 3a,6,6,9a-tetramethyldodecahydronaphtho[2,1-b]furan; 3a-ethyl-6,6,9a-trimethyldodecahydronaphtho[2,1-b]furan; 1,5,9-trimethyl-13-oxabicyclo[10.1.0]-trideca-4,8-diene; rose oxide; 2-(2,4-dimethyl-3-cyclohexen-1-yl)-5-methyl-5-(1-methylpropyl)-1,3-dioxane;

the cyclic ketones, such as e.g. 4-tert-butylcyclohexanone; 2,2,5-trimethyl-5-pentylcyclopentanone; 2-heptylcyclopentanone; 2-pentylcyclopentanone; 2-hydroxy-3-methyl-2-cyclopenten-1-one; 3-methyl-cis-2-penten-1-yl-2-cyclopenten-1-one; 3-methyl-2-pentyl-2-cyclopenten-1-one; 3-methyl-4-cyclopentadecenone; 3-methyl-5-cyclopentadecenone; 3-methylcyclopenta-decanone; 4-(1-ethoxyvinyl)-3,3,5,5-tetramethylcyclohexanone; 4-tert-pentyl-cyclohexanone; 5-cyclohexadecen-1-one; 7-cyclohexadecen-1-one; 8-cyclohexadecen-1-one; 6,7-dihydro-1,1,2,3,3-pentamethyl-4(5H)-indanone; 9-cycloheptadecen-1-one; cyclopentadecanone; cyclohexadecanone;

the cycloaliphatic aldehydes, such as e.g. 2,4-dimethyl-3-cyclohexenecarbaldehyde; 2-methyl-4-(2,2,6-trimethyl-cyclohexen-1-yl)-2-butenal; 4-(4-hydroxy-4-methylpentyl)-3-cyclohexenecarbaldehyde; 4-(4-methyl-3-penten-1-yl)-3-cyclohexenecarbaldehyde;

the cycloaliphatic ketones, such as e.g. 1-(3,3-dimethylcyclohexyl)-4-penten-1-one; 1-(5,5-dimethyl-1-cyclohexen-1-yl)-4-penten-1-one; 2,3,8,8-tetramethyl-1,2,3,4,5,6,7,8-octahydro-2-naphthalenyl methyl ketone; methyl 2,6,10-trimethyl-2,5,9-cyclododecatrienyl ketone; tert-butyl(2,4-dimethyl-3-cyclohexen-1-yl)ketone;

the esters of cyclic alcohols, such as e.g. 2-tert-butylcyclohexyl acetate; 4-tert-butylcyclohexyl acetate; 2-tert-pentylcyclohexyl acetate; 4-tert-pentylcyclohexyl acetate; decahydro-2-naphthyl acetate; 3-pentyltetrahydro-2H-pyran-4-yl acetate; decahydro-2,5,5,8a-tetramethyl-2-naphthyl acetate; 4,7-methano-3a,4,5,6,7,7a-hexahydro-5- and -6-indenyl acetate; 4,7-methano-3a,4,5,6,7,7a-hexahydro-5- and -6-indenyl propionate; 4,7-methano-3a,4,5,6,7,7a-hexahydro-5- and -6-indenyl isobutyrate; 4,7-methanooctahydro-5- and 6-indenyl acetate;

the esters of cycloaliphatic carboxylic acids, such as e.g. allyl 3-cyclohexylpropionate; allyl cyclohexyloxyacetate; methyl dihydrojasmonate; methyl jasmonate; methyl 2-hexyl-3-oxocyclopentanecarboxylate; ethyl 2-ethyl-6,6-dimethyl-2-cyclohexenecarboxylate; ethyl 2,3,6,6-tetramethyl-2-cyclohexene-carboxylate; ethyl 2-methyl-1,3-dioxolane-2-acetate;

the aromatic hydrocarbons, such as e.g. styrene and diphenylmethane;

the araliphatic alcohols, such as e.g. benzyl alcohol; 1-phenylethyl alcohol; 2-phenylethyl alcohol; 3-phenylpropanol; 2-phenylpropanol; 2-phenoxyethanol; 2,2-dimethyl-3-phenylpropanol; 2,2-dimethyl-3-(3-methylphenyl)propanol; 1,1-dimethyl-2-phenylethyl alcohol; 1,1-dimethyl-3-phenylpropanol; 1-ethyl-1-methyl-3-phenylpropanol; 2-methyl-5-phenylpentanol; 3-methyl-5-phenylpentanol; 3-phenyl-2-propen-1-ol; 4-methoxybenzyl alcohol; 1-(4-isopropylphenyl)ethanol;

the esters of araliphatic alcohols and aliphatic carboxylic acids, such as e.g.: benzyl acetate; benzyl propionate; benzyl isobutyrate; benzyl isovalerate; 2-phenylethyl acetate; 2-phenylethyl propionate; 2-phenylethyl isobutyrate; 2-phenylethyl isovalerate; 1-phenylethyl acetate; alpha-trichloromethylbenzyl acetate; alpha,alpha-dimethylphenylethyl acetate; alpha,alpha-dimethylphenylethyl butyrate; cinnamyl acetate; 2-phenoxyethyl isobutyrate; 4-methoxybenzyl acetate; the araliphatic ethers, such as e.g. 2-phenylethyl methyl ether; 2-phenylethyl isoamyl ether; 2-phenylethyl 1-ethoxyethyl ether; phenylacetaldehyde dimethyl acetal; phenylacetaldehyde diethyl acetal; hydratropaaldehyde dimethyl acetal; phenylacetaldehyde glycerol acetal; 2,4,6-trimethyl-4-phenyl-1,3-dioxane; 4,4a,5,9b-tetrahydroindeno[1,2-d]-m-dioxin; 4,4a,5,9b-tetrahydro-2,4-dimethylindeno[1,2-d]-m-dioxin;

the aromatic and araliphatic aldehydes, such as e.g. benzaldehyde; phenylacetaldehyde; 3-phenylpropanal; hydratropaaldehyde; 4-methylbenzaldehyde; 4-methylphenylacetaldehyde; 3-(4-ethylphenyl)-2,2-dimethylpropanal; 2-methyl-3-(4-isopropylphenyl)propanal; 2-methyl-3-(4-tert-butylphenyl)propanal; 3-(4-tert-butylphenyl)propanal; cinnamaldehyde; alpha-butylcinnamaldehyde; alpha-amylcinnamaldehyde; alpha-hexylcinnamaldehyde; 3-methyl-5-phenylpentanal; 4-methoxybenzaldehyde; 4-hydroxy-3-methoxybenzaldehyde; 4-hydroxy-3-ethoxybenzaldehyde; 3,4-methylene-dioxybenzaldehyde; 3,4-dimethoxybenzaldehyde; 2-methyl-3-(4-methoxy-phenyl)propanal; 2-methyl-3-(4-methylenedioxyphenyl)propanal;

the aromatic and araliphatic ketones, such as e.g. acetophenone; 4-methyl-acetophenone; 4-methoxyacetophenone; 4-tert-butyl-2,6-dimethylacetophenone; 4-phenyl-2-butanone; 4-(4-hydroxyphenyl)-2-butanone; 1-(2-naphthalenyl)-ethanone; benzophenone; 1,1,2,3,3,6-hexamethyl-5-indanyl methyl ketone; 6-tert-butyl-1,1-dimethyl-4-indanyl methyl ketone; 1-[2,3-dihydro-1,1,2,6-tetramethyl-3-(1-methylethyl)-1H-5-indenyl]ethanone; 5′,6′,7′,8′-tetrahydro-3′,5′,5′,6′,8′,8′-hexamethyl-2-acetonaphthone;

the aromatic and araliphatic carboxylic acids and esters thereof, such as e.g. benzoic acid; phenylacetic acid; methyl benzoate; ethyl benzoate; hexyl benzoate; benzyl benzoate; methylphenyl acetate; ethylphenyl acetate; geranylphenyl acetate; phenylethyl-phenyl acetate; methyl cinnamate; ethyl cinnamate; benzyl cinnamate; phenylethyl cinnamate; cinnamyl cinnamate; allyl phenoxyacetate; methyl salicylate; isoamyl salicylate; hexyl salicylate; cyclohexyl salicylate; cis-3-hexenyl salicylate; benzyl salicylate; phenylethyl salicylate; methyl 2,4-dihydroxy-3,6-dimethylbenzoate; ethyl 3-phenylglycidate; ethyl 3-methyl-3-phenylglycidate;

the nitrogen-containing aromatic compounds, such as e.g. 2,4,6-trinitro-1,3-dimethyl-5-tert-butylbenzene; 3,5-dinitro-2,6-dimethyl-4-tert-butylacetophenone; cinnamic acid nitrile; 5-phenyl-3-methyl-2-pentenoic acid nitrile; 5-phenyl-3-methylpentanoic acid nitrile; methyl anthranilate; methyl N-methylanthranilate; Schiff's bases of methyl anthranilate with 7-hydroxy-3,7-dimethyloctanal, 2-methyl-3-(4-tert-butylphenyl)propanal or 2,4-dimethyl-3-cyclohexene-carbaldehyde; 6-isopropylquinoline; 6-isobutylquinoline; 6-sec-butylquinoline; indole; skatole; 2-methoxy-3-isopropylpyrazine; 2-isobutyl-3-methoxypyrazine;

the phenols, phenyl ethers and phenyl esters, such as e.g. estragole; anethole; eugenol; eugenyl methyl ether; isoeugenol; isoeugenyl methyl ether; thymol; carvacrol; diphenyl ether; beta-naphthyl methyl ether; beta-naphthyl ethyl ether; beta-naphthyl isobutyl ether; 1,4-dimethoxybenzene; eugenyl acetate; 2-methoxy-4-methylphenol; 2-ethoxy-5-(1-propenyl)phenol; p-cresyl phenylacetate;

the heterocyclic compounds, such as e.g. 2,5-dimethyl-4-hydroxy-2H-furan-3-one; 2-ethyl-4-hydroxy-5-methyl-2H-furan-3-one; 3-hydroxy-2-methyl-4H-pyran-4-one; 2-ethyl-3-hydroxy-4H-pyran-4-one;

the lactones, such as e.g. 1,4-octanolide; 3-methyl-1,4-octanolide; 1,4-nonanolide; 1,4-decanolide; 8-decen-1,4-olide; 1,4-undecanolide; 1,4-dodecanolide; 1,5-decanolide; 1,5-dodecanolide; 1,15-pentadecanolide; cis- and trans-11-pentadecen-1,15-olide; cis- and trans-12-pentadecen-1,15-olide; 1,16-hexadecanolide; 9-hexadecen-1,16-olide; 10-oxa-1,16-hexadecanolide; 11-oxa-1,16-hexadecanolide; 12-oxa-1,16-hexadecanolide; ethylene 1,12-dodecanedioate; ethylene 1,13-tridecanedioate; coumarin; 2,3-dihydrocoumarin; octahydrocoumarin.

In perfume oil compositions (=odoriferous substance mixtures) according to the invention which comprise e.g. abovementioned other perfume constituents, the amount of 2-methyl-2-alkenyl-substituted 1,3-dioxanes of the formula (I) employed is usually in the range of from 0.001 to 50 wt. %, preferably 0.05 to 30 wt. % and particularly preferably 0.5 to 20 wt. %, based on the total amount of odoriferous substances contained in the perfume oil composition.

Perfume oil compositions (=perfume oils) which comprise 2-methyl-2-alkenyl-substituted 1,3-dioxanes of the formula (I) can be employed (as a perfumed product) for perfumings in liquid form, undiluted or diluted with a solvent. Suitable solvents for this are e.g. ethanol, isopropanol, diethylene glycol monoethyl ether, glycerol, propylene glycol, 1,2-butylene glycol, dipropylene glycol, diethyl phthalate, triethyl citrate, isopropyl myristate. In the context of the present text, the solvents mentioned are not interpreted as odoriferous substances.

For some uses, it is advantageous to employ perfume oils (odoriferous substance mixtures) comprising compounds of the formula (I) according to the invention which are adsorbed on a carrier substance, which ensures both a fine distribution of the odoriferous substances in the product and a controlled release during use. Such carriers can be porous inorganic materials, such as light sulfate, silica gels, zeolites, gypsums, clays, clay granules, gas concrete etc., or organic materials, such as woods; cellulose-based substances, sugars or plastics, such as PVC, polyvinyl acetates or polyurethanes.

For other uses, it is advantageous to employ perfume oils which comprise compounds of the formula (I) according to the invention in microencapsulated or spray-dried form or in the form of an inclusion complex or extrusion product and to add them in this form to the precursor/product to be perfumed.

The properties of perfume oil compositions according to the invention modified in this manner are in some cases optimized further in respect of a more controlled release of fragrance by so-called “coating” with suitable materials, for which purpose wax-like plastics, such as e.g. polyvinyl alcohol, are preferably used.

The microencapsulation of the perfume oils can be carried out, for example, by the co-called coacervation process with the aid of capsule materials e.g. of polyurethane-like substances or soft gelatine. The spray-dried perfume oils can be prepared, for example, by spray drying of an emulsion or dispersion containing the perfume oil, it being possible to use modified starches, proteins, dextrin and plant gums as carrier substances. Inclusion complexes can be prepared e.g. by introducing dispersions of the perfume oil and cyclodextrins or urea derivatives into a suitable solvent, e.g. water. Extrusion products can be achieved by melting the perfume oils with a suitable wax-like substance and by extrusion with subsequent solidification, optionally in a suitable solvent, e.g. isopropanol.

Ingredients with which compounds of the formula (I), mixtures and perfume oil compositions according to the invention can be combined are, for example: preservatives, abrasives, antiacne agents, agents against ageing of the skin, antibacterial agents, anticellulitis agents, antidandruff agents, antiinflammatory agents, irritation-preventing agents, irritation-inhibiting agents, antimicrobial agents, antioxidants, astringents, perspiration-inhibiting agents, antiseptic agents, antistatics, binders, buffers, carrier materials, chelating agents, cell stimulants, cleansing agents, care agents, depilatory agents, surface-active substances, deodorizing agents, antiperspirants, softeners, emulsifiers, enzymes, essential oils, fibres, film-forming agents, fixatives, foam-forming agents, foam stabilizers, substances for preventing foaming, foam boosters, fungicides, gelling agents, gel-forming agents, hair care agents, hair setting agents, hair straightening agents, moisture-donating agents, moisturizing substances, moisture-retaining substances, bleaching agents, strengthening agents, stain-removing agents, optically brightening agents, impregnating agents, dirt-repellent agents, friction-reducing agents, lubricants, moisturizing creams, ointments, opacifying agents, plasticizing agents, covering agents, polish, gloss agents, polymers, powders, proteins, re-oiling agents, abrading agents, silicones, skin soothing agents, skin cleansing agents, skin care agents, skin healing agents, skin lightening agents, skin protecting agents, skin softening agents, cooling agents, skin cooling agents, warming agents, skin warming agents, stabilizers, UV-absorbing agents, UV filters, detergents, laundry softeners, suspending agents, skin tanning agents, thickeners, vitamins, oils, waxes, fats, phospholipids, saturated fatty acids, mono- or polyunsaturated fatty acids, a-hydroxy acids, polyhydroxy-fatty acids, liquefiers, dyestuffs, colour-protecting agents, pigments, anticorrosives, aromas, flavouring substances, odoriferous substances, polyols, surfactants, electrolytes, organic solvents or silicone derivatives.

Perfume oils according to the invention comprising 2-methyl-2-alkenyl-substituted 1,3-dioxanes of the formula (I) can be used in concentrated form, in solutions or in an otherwise modified form for the preparation of e.g. perfume extracts, perfume waters, toilet waters, shaving lotions, cologne waters, pre-shave products, splash colognes and perfumed freshening wipes, as well as perfuming of acid, alkaline and neutral cleaning compositions, such as e.g. floor cleaners, window glass cleaners, dishwashing compositions, bath and sanitary cleaners, scouring milk, solid and liquid WC cleaners, powder and foam carpet cleaners, liquid detergents, pulverulent detergents, laundry pretreatment compositions, such as bleaching compositions, soaking compositions and stain removers, laundry softeners, washing soaps, washing tablets, disinfectants, surface disinfectants and air fresheners in liquid or gelatinous form or in a form applied to a solid carrier, aerosol sprays, waxes and polishes, such as furniture polishes, floor waxes, shoe creams as well as body care compositions, such as e.g. solid and liquid soaps, shower gels, shampoos, shaving soaps, shaving foams, bath oils, cosmetic emulsions of the oil-in-water, of the water-in-oil and of the water-in-oil-in-water type, such as e.g. skin creams and lotions, face creams and lotions, sunscreen creams and lotions, after-sun creams and lotions, hand creams and lotions, foot creams and lotions, depilatory creams and lotions, after-shave creams and lotions, tanning creams and lotions, hair care products, such as e.g. hair sprays, hair gels, hair lotions, hair conditioners, permanent and semi-permanent hair colouring compositions, hair setting compositions, such as cold waving compositions and hair straightening compositions, hair waters, hair creams and lotions, deodorants and antiperspirants, such as e.g. underarm sprays, roll-ons, deodorant sticks, deodorant creams or products for decorative cosmetics.

Particularly preferred perfumed products according to the invention are detergents and hygiene or care products, in particular in the field of body care, cosmetics and household products.

Odoriferous substance mixtures with a damascone-like, fruity and fresh (head) note are furthermore often sought-after—in particular for perfuming surfactant-containing formulations, such as, for example, for shampoos, detergents or laundry softeners—and these should simultaneously have a pronounced blooming (smell from an aqueous surfactant solution).

The 2-methyl-2-alkenyl-substituted 1,3-dioxanes of the formula (I) according to the invention and the abovementioned mixtures according to the invention which additionally comprise, in addition to one or more compounds of the formula (I), the compound of the formula (IV) and/or (V) have particular olfactory effects especially in body care compositions, specifically in shampoos, and in household products, specifically in laundry softeners (see also the following examples). In particular, they are capable of imparting and/or intensifying a damascone-like, fruity and fresh (head) note, a pronounced blooming (smell from an aqueous surfactant solution) being observed at the same time.

It has furthermore been found that the compounds of the formula (I) according to the invention and the mixtures according to the invention have a high impact and a high popularity, in particular as a constituent of a shampoo or laundry softener.

By a content of one or more compounds of the formula (I) in a perfume oil composition, preferably in a total amount in the range of from 0.5 to 4 wt. %, based on the total weight of the perfume oil composition, in particular in perfume oil compositions for use in shampoo or a laundry softener, the damascone-like, fruity and fresh character is increased significantly compared with the starting composition, and at the same time a pronounced blooming is obtained. This effect occurs more intensively—in particular the damascone-like aspect—at a content of a mixture according to the invention in the same amount, and the perfume oil composition acquires an even more complex olfactory impression.

The compounds of the formula (I) and the abovementioned mixtures according to the invention furthermore also act as so-called boosters or enhancers, i.e. they have the effect of intensifying the smell or the olfactory perception of other odoriferous substances in odoriferous substance mixtures and perfume compositions.

Compounds of the formula (I) and the abovementioned mixtures according to the invention intensify in particular the fresh character (very potent boosters) in a perfume oil composition for laundry softeners. A fresh effect is of great importance for perfume oil compositions in the laundry softening field. This effect is perceived in particular when the compounds of the formula (I) are employed in a total amount of from 0.5 wt. % to 4 wt. %, based on the total weight of the perfume oil composition.

The following examples illustrate the invention; unless stated otherwise, contents and percentages relate to the weight.

### EXAMPLE 1

### General Working Instructions for the Preparation of the Compounds of the Formula (I)

0.37 mol aliphatic 1,3-diol (D) in cyclohexane with 0.0003 mol para-toluenesulfonic acid is added to 0.31 mol ketone (K) and the mixture is boiled under reflux for at least two hours, using a water separator. The reaction mixture is washed thoroughly first with 5 wt. % strength sodium carbonate solution and then with water. The organic phase is dried over potassium carbonate, concentrated, and distilled in vacuo.

### EXAMPLE 1.1

### 2,5-Dimethyl-2-(1′-methyl-propenyl)-1,3-dioxane

2,5-Dimethyl-2-(1′-methyl-propenyl)-1,3-dioxane according to the invention was prepared in accordance with the above general working instructions from 0.31 mol 3-methyl-3-penten-2-one (compound of the formula (V)) and 0.37 mol 2-methyl-1,3-propanediol (50% of theory, purity: >98% according to GC; main isomer >95 GC-%, secondary isomer >3 GC-%).

Main Isomer:

¹H-NMR (400 MHz, CDCl₃): δ (ppm)=0.65 (d, J=6.68 Hz, 3H), 1.33 (s, 3H), 1.59-1.61 (m, 3H), 1.66-1.69 (m, 3H), 1.99-2.10 (m, 1H), 3.26-3.33 (m, 2H), 3.64-3.70 (m, 2H), 5.69 (dq, J=6.19, 1.45 Hz, 1H).

¹³C-NMR (101 MHz, CDCl₃): δ (ppm)=12.01, 12.56, 13.47, 28.63, 28.97, 67.39 (2C), 100.91, 123.01, 133.66

Secondary Isomer:

¹H-NMR (400 MHz, CDCl₃): δ (ppm)=1.16 (d, J=6.91 Hz, 3H), 1.37 (s, 3H), 1.62-1.63 (m, 3H), 1.65-1.67 (m, 3H), 1.99-2.10 (m, 1H), 3.47-3.52 (m, 2H), 3.87-3.92 (m, 2H), 5.69 (dq, J=6.19, 1.45 Hz, 1H).

¹³C-NMR (101 MHz, CDCl₃): δ (ppm)=11.87, 13.38, 15.11, 25.81, 28.73, 66.11 (2C), 100.66, 122.02, 134.75

### EXAMPLE 1.2

### 2,5,5-Trimethyl-2-(1′-methyl-propenyl)-1,3-dioxane

2,5,5-Trimethyl-2-(1′-methyl-propenyl)-1,3-dioxane according to the invention was prepared in accordance with the above general working instructions from 0.31 mol 3-methyl-3-penten-2-one (compound of the formula (V)) and 0.37 mol 2.2-dimethyl-1,3-propanediol (72% of theory, purity: >99% according to GC, 1 peak, only (E) isomer).

¹H-NMR (400 MHz, CDCl₃): δ (ppm)=0.68 (s, 3H), 1.19 (s, 3H), 1.36 (s, 3H), 1.58-1.61 (m, 3H), 1.65-1.68 (m, 3H), 3.26-3.30 (m, 2H), 3.41-3.46 (m, 2H), 5.65-5.72 (m, 1H).

¹³C-NMR (101 MHz, CDCl₃): δ (ppm)=11.93, 13.45, 22.08, 22.78, 28.05, 29.58, 71.40 (2C) 100.94, 122.73, 133.59.

### EXAMPLE 2

### Perfume Oil, e.g. for Use in Shampoo

Preparation of a perfume oil having a modern, distinctly fresh, fruity and damascone-like note.

The following odoriferous substances are mixed in the stated amounts:

| | Parts by weight |
|---|---|
| Agrumex (2-tert-butylcyclohexyl acetate) | 9 |
| Aldehyde C 14, so-called | 5 |
| Alcohol C 6, kosher | 2 |
| Allyl caproate, kosher 10% strength in DPG | 1 |
| Benzyl formate | 1.5 |
| Butyl acetate | 0.5 |
| Dibenzyl ether | 1.5 |
| Dipropylene glycol | 37.5 |
| Ethyl butyrate | 0.5 |
| Ethyl trans,cis-2,4-decadienoate | 1 |
| Ethyl 2-methylbutyrate | 0.5 |
| Exaltolide | 2 |
| Geranyl acetate | 10 |
| Globalide ® (11(12)-pentadecen-15-olide) | 6 |
| cis-3-Hexenol | 0.5 |
| trans-2-hexenol | 0.5 |
| cis-3-Hexenyl acetate | 2 |
| Hexyl acetate | 5 |
| Isoamyl acetate | 2.5 |
| Ligustral | 0.5 |
| Maltol | 0.5 |
| Neryl acetate | 6 |
| Parmanyl ® | 1.5 |
| [3-(cis-3-hexenyloxy)-propanenitrile] | |
| Prenyl acetate | 1.5 |
| Total | 98.5 |

DPG: Dipropylene glycol

This starting composition is a perfume oil with a fruity smell with a distinct apple and pear note.

### EXAMPLE 2.1

By addition of 1.5 parts by weight of 2,5,5-trimethyl-2-(1′-methyl-propenyl)-1,3-dioxane from Example 1.2, a modern perfume oil having a distinctly fresh and damascone-like note is obtained. Compared with the starting composition, the fruity character is also intensified noticeably, and in particular the pear-like note is intensified. The odoriferous substance composition obtained according to the invention is more harmonious and rounded-off, and the radiance and spatial effect (diffusivity) are likewise increased compared with the starting composition.

### EXAMPLE 2.2

On addition of 1.5 parts by weight of a mixture consisting of 96 wt. % 2,5,5-trimethyl-2-(1′-methyl-propenyl)-1,3-dioxane from Example 1.2 and 4 wt. % 2-ethyl-2,5,5-trimethyl-1,3-dioxane of the formula (IV) to the starting composition, the effects described under Example 2.1 are intensified further and the perfume oil acquires an even more complex olfactory impression.

### EXAMPLE 3

### Shampoo

The perfume oil composition from Example 2.2 (after addition of 1.5 parts by weight of 2,5,5-trimethyl-2-(1′-methyl-propenyl)-1,3-dioxane from Example 1.2) was incorporated in a dosage of 0.5 wt. % into a shampoo base mass of the following composition:

| | |
|---|---|
| Sodium lauryl ether-sulfate | 12% |
| (e.g. Texapon NSO, Cognis Deutschland GmbH) | |
| Cocamidopropylbetaine | 2% |
| (e.g. Dehyton K, Cognis Deutschland GmbH) | |
| Sodium chloride | 1.4% |
| Citric acid | 1.3% |
| Phenoxyethanol, methyl-, ethyl-, butyl- | 0.5% |
| and propylparaben | |
| Water | 82.8% |

The pH of the shampoo base mass was about 6. 100 ml of a 20 wt. % strength aqueous shampoo solution were prepared from this. The shampoo solution displayed pronounced blooming. 2 hanks of hair were washed together in this shampoo solution for 2 minutes and were then rinsed for 20 seconds under running hand-hot water. One hank of hair was packed in aluminium foil while wet and the second hank of hair was dried with a hair-dryer. The olfactory properties of the two hanks of hair were evaluated by a panel. Both hanks of hair displayed a fruity-fresh, slightly damascone-like smell, the overall impression being perceived as radiant, rounded-off and harmonious.

### EXAMPLE 4

### Perfume Oil, e.g. for Use in a Laundry Softener

Preparation of a perfume oil having a modern, distinctly flowery-fresh note.

The following odoriferous substances are mixed in the stated amounts:

| | Parts by weight |
|---|---|
| Acetophenone, 10% strength in DPG | 1 |
| Undecanal | 0.5 |
| Aldehyde C14, so-called (peach aldehyde) | 1.5 |
| Allyl amyl glycolate, 10% strength in DPG | 2 |
| n/iso-Amyl salicylate | 2.5 |
| Citronellol | 12 |
| trans-9-Decenol | 0.5 |
| Dihydromyrcenol | 3 |
| Dimethylbenzylcarbinyl acetate | 3 |
| Diphenyl oxide | 0.5 |
| Eugenol | 1 |
| Geraniol | 6 |
| Egyptian geranium oil | 1.5 |
| cis-3-Hexenol, 10% strength in DPG | 0.5 |
| cis-3-Hexenyl salicylate | 2 |
| Indole, 10% strength in DPG | 1 |
| alpha-Ionone | 1.5 |
| beta-Ionone | 0.5 |
| Isoeugenol | 0.5 |
| Isoraldein | 1.5 |
| Lilial | 6 |
| Linalool | 4 |
| Methylphenyl acetate | 1 |
| Brazilian orange oil | 1 |
| Palmarosa oil | 0.5 |
| Patchouli oil | 0.5 |
| Phenylethyl alcohol | 27.5 |
| Rose oxide, 10% strength in DPG | 2 |
| Styrenyl acetate | 2 |
| Terpineol | 3 |
| Tetrahydrolinalool | 5 |
| Vanillin, 10% strength in DPG | 0.5 |
| Vertocitral | 0.5 |
| Cinnamyl alcohol | 1 |
| Total | 97 |

DPG: Dipropylene glycol

This starting composition is a perfume oil for laundry softeners having a flowery accord with a wild rose note.

### EXAMPLE 4.1

By addition of 1.5 parts by weight of 2,5-dimethyl-2-(1′-methyl-propenyl)-1,3-dioxane from Example 1.1 and 1.5 parts by weight of 2,5,5-trimethyl-2-(1′-methyl-propenyl)-1,3-dioxane from Example 1.2 to the starting composition, a modern perfume (=product according to the invention) having a distinctly flowery-fresh laundry softening note is obtained. Compared with the starting composition, a significantly more intense, fresher more flowery smell (very high intensification (booster)) with a slightly damascone-like note is perceived. The product according to the invention is more harmonious and rounded-off, and the radiance and spatial effect (diffusivity) are also increased compared with the starting composition.

### EXAMPLE 4.2

On addition of 1.5 parts by weight of a mixture consisting of 95.8 wt. % 2,5,5-trimethyl-2-(1′-methyl-propenyl)-1,3-dioxane from Example 1.2, 3.8 wt. % 2-ethyl-2,5,5-trimethyl-1,3-dioxane of the formula (IV) and 0.4 wt. % 3-methyl-3-penten-2-one of the formula (V) to the starting composition, the effects described under Example 4.1 are intensified further and the perfume oil acquires an even more complex olfactory impression.

### EXAMPLE 5

### Laundry Softener

The perfume oil composition from Example 4.1 (after addition of 2,5-dimethyl-2-(1′-methyl-propenyl)-1,3-dioxane and 2,5,5-trimethyl-2-(1′-methyl-propenyl)-1,3-dioxane in the stated proportions) was incorporated in a dosage of 0.5 wt. % into a laundry softener base mass of the following composition:

| | |
|---|---|
| Quaternary ammonium methosulfate (ester quat), approx. 90% | 5.5% |
| (e.g. Rewoquat WE 18, Witco Surfactants GmbH) | |
| Alkyldimethylbenzylammonium chloride, approx. 50% | 0.2% |
| (e.g. Preventol R50, Bayer AG) | |
| Colouring solution, approx. 1% strength | 0.3% |
| Water | 94.0% |

The pH of the laundry softener base mass was in the range of 2-3. Two fabric rags were washed with 370 g of a 1% strength aqueous laundry softener solution in a Linetest machine in the softening programme for 30 minutes at 20° C. The rags were wrung out and then spun for 20 seconds. One rag was sealed while wet, and one was hung up to dry. The olfactory properties of the two rags were then evaluated by a panel. Both rags displayed a fruity-flowery, slightly damascone-like smell, the overall impression being perceived as radiant, rounded-off and harmonious.

What is claimed is:

1. A compound of the formula (I) 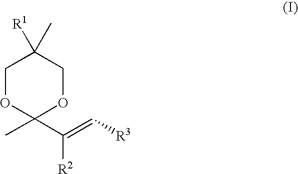
wherein
R¹ denotes hydrogen or methyl, and R² and R³ independently of one another denote methyl or ethyl.

2. The compound according to claim 1, wherein R² denotes methyl.

3. The compound according to claim 1, wherein R² and R³ each denote methyl.

4. The compound according to claim 1, wherein the double bond has the (E) configuration.

5. The compound according to claim 1, selected from the group consisting of:
(E)-2,5-dimethyl-2-(1′-methyl-propenyl)-1,3-dioxane
and
(E)-2,5,5-trimethyl-2-(1′-methyl-propenyl)-1,3-dioxane.

6. A mixture comprising:
(a) one or more compounds according to claim 1
and
(b) 2-ethyl-2,5,5-trimethyl-1,3-dioxane of the formula (IV) 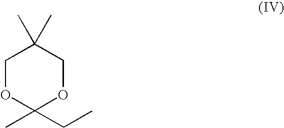
and/or
(c) 3-methyl-3-penten-2-one of the formula (V) 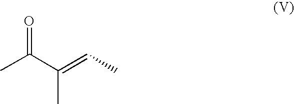
and optionally one, two or more further odoriferous substances.

7. The mixture comprising:
(a) 78.0 to 99.9 parts by weight in total of one or more compounds according to claim 1,
and
(b) 0.25 to 20 parts by weight of 2-ethyl-2,5,5-trimethyl-1,3-dioxane of the formula (IV)
and/or
(c) 0.1 to 3.0 parts by weight of 3-methyl-3-penten-2-one of the formula (V), wherein the sum of constituents (a), (b) and (c) is 100 parts by weight, and optionally one, two or more further odoriferous substances.

8. The mixture comprising:
(a) 91 to 99.75 parts by weight in total of one or more compounds according to claim 1,
and
(b) 0.5 to 8 parts by weight of 2-ethyl-2,5,5-trimethyl-1,3-dioxane of the formula (IV)
and/or
(c) 0.25 to 1 part by weight of 3-methyl-3-penten-2-one of the formula (V), wherein the sum of constituents (a), (b) and (c) is 100 parts by weight, and optionally one, two or more further odoriferous substances.

9. The mixture according to claim 6, wherein the total amount of the compound according to claim 1 employed is in the range of from 0.001 to 50 wt. % based on the total amount of odoriferous substances contained in the mixture.

10. The mixture according to claim 6, wherein the total amount of the compound of claim 1 is 0.05 to 30 wt. % based on the total amount of the odoriferous substances.

11. The mixture of claim 6, wherein the total amount of the compound of claim 1 is 0.5 to 20 wt. % based on the total amount of odoriferous substance.

12. An odoriferous substance or booster comprising a compound of claim 1.

13. An odoriferous substance, booster or perfumed product comprising a mixture according to claim 6.

14. A process for the preparation of a compound according to claim 1, with the following steps: reaction of a ketone of the formula (K) 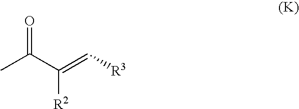
wherein R² and R³ each have the meaning mentioned in claim 1,
with an aliphatic 1,3-diol of the formula (D) 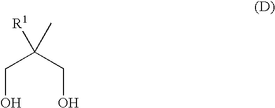
wherein R¹ has the meaning mentioned in claim 1,
preferably in the presence of an acid catalyst.

15. A perfumed product, comprising
(A) a solid or semi-solid carrier
and
a sensorially active amount, in contact with the solid or semi-solid carrier, of a
compound according to claim 1
or
mixture of a compound of claim 1, and 2-ethyl-2,5,5-trimethyl-1,3-dioxane and/or 3-methyl-3-penten-2-one,
or
(B) a liquid phase
and
dissolved or suspended therein or diluted therewith, a sensorially active amount of
a compound according to claim 1
or
mixture of a compound of claim 1, and 1,2-ethyl-2,5,5-trimethyl-1,3-dioxane and/or 3-methyl-3-penten-2-one.

16. The perfumed product according to claim 15, selected from the group consisting of: perfume extracts, perfume waters, toilet waters, shaving lotions, cologne waters, pre-shave products, splash colognes, perfumed freshening wipes, acid, alkaline and neutral cleaning compositions, textile fresheners, ironing aids, liquid detergents, pulverulent detergents, laundry pretreatment compositions, laundry softeners, washing soaps, washing tablets, disinfectants, surface disinfectants, air fresheners, aerosol sprays, waxes and polishes, body care compositions, hand creams and lotions, foot creams and lotions, depilatory creams and lotions, after-shave creams and lotions, tanning creams and lotions, hair care products, deodorants and antiperspirants, products for decorative cosmetics, candles, lamp oils, joss sticks, insecticides, repellents and fuels.

17. The perfumed product according to claim 15, selected from the group consisting of: shampoos and laundry softeners.

18. A method of imparting, modifying and/or intensifying a smell, having the following step:
bringing into contact or mixing of a product with a sensorially active amount of a
compound according to claim 1
or
mixture of a compound of claim 1, and 1,2-ethyl-2,5,5-trimethyl-1,3-dioxane and/or 3-methyl-3-penten-2-one.
